(19) United States
(12) Patent Application Publication
(10) Pub. No.: US 2025/0029253 A1
(43) Pub. Date: Jan. 23, 2025

(54) METHOD AND SYSTEM FOR DETECTION OF HISTOPATHOLOGICAL PLAQUE FEATURES IN MEDICAL IMAGES USING DEEP NEURAL NETWORKS

(71) Applicant: Styliani Stella Daskalopoulou, Westmount (CA)

(72) Inventor: Styliani Stella Daskalopoulou, Westmount (CA)

(21) Appl. No.: 18/707,365

(22) PCT Filed: Nov. 4, 2022
(86) PCT No.: PCT/CA2022/051639

§ 371 Date: May 3, 2024

Related U.S. Application Data
(60) Provisional application No. 63/276,015, filed on Nov. 5, 2021.

(51) Int. Cl. G06T 7/00; A61B 8/08; G06T 3/40; G06T 7/30; G06V 10/26; G06V 10/82

(52) U.S. Cl. CPC G06T 7/0014; A61B 8/5223; A61B 8/5292; G06T 3/40; G06T 7/30; G06V 10/26; G06V 10/82; G06T 2207/10056; G06T 2207/10132; G06T 2207/20081; G06T 2207/20084; G06T 2207/30024; G06T 2207/30101; G06V 2201/03

(57) ABSTRACT

A method of training a neural network for segmenting an information-poor image to identify a plurality of atherosclerotic plaque features in the information-poor image; it has receiving a plurality of image pairs, wherein each image pair comprises an information-poor image of specific vasculature and an information-rich image of the specific vasculature; performing image registration to map the information-poor image and the information-rich image into a same coordinate system; segmenting the information-poor image as a function of the identified one or more regions of the information-rich image, thereby identifying in the information-poor image the one or more plaque features; and comparing the segmented information-poor image to a ground truth based on the information-rich image to calculate a loss that is back-propagated through the neural network to train the neural network.

The present application claims priority from U.S. provisional patent application No. 63/276,015 filed on Nov. 5, 2021, incorporated herein by reference.

### TECHNICAL FIELD

The present disclosure relates to methods of detection of atherosclerotic plaque features, and more particularly to methods of detection of atherosclerotic plaque features in information-poor images in order to assess the risk of vascular events in subjects.

### BACKGROUND

Strokes and heart attacks are the leading causes of death and long-term disability world-wide, caused by the rupture of unstable atherosclerotic plaques (also known as high-risk or vulnerable plaques) in the arteries of the neck and heart. Current clinical guidelines recommend surgical intervention of plaques (removal or stenting) based solely on the degree of artery stenosis caused by the plaque. However, it is increasingly recognized that stenosis alone is an incomplete determinant of heart attack or stroke risk, as it does not entirely reflect a plaque's true instability and likelihood to rupture, leading to suboptimal medical decisions and inappropriate treatment allocation. Many plaques causing high-grade stenoses remain stable and asymptomatic, while unstable and potentially dangerous plaques often cause moderate or even low-grade stenoses. As a result, while many subjects with stable plaques are being recommended unnecessary surgeries that impose unjustified subject risk and a burden to the healthcare system, others with unstable plaques that are more likely to rupture are not receiving proper treatment. Instead, plaque morphology and composition are more accurate indicators of plaque instability and better predictors of clinical outcomes compared to stenosis alone.

Histology is the gold-standard method for assessing atherosclerotic plaque stability/instability. Plaques have a complex composition, consisting of an accumulation of inflammatory cells, smooth muscle cells, fibrous tissue, lipids, cholesterol crystals, hemorrhage, and calcification. Due to their heterogeneity, plaques can either be classified as stable or unstable based on the presence/extent of certain histological features. Unstable plaques are characterized by a large lipid-rich core, a thin fibrous cap, a chronic inflammatory state, and intraplaque hemorrhage, thrombus, and are highly prone to rupture or have ruptured. In contrast, stable plaques have a thick fibrous cap, which protects them from rupturing, and little-to-no presence of a lipid core. Currently, researchers rely on qualitative/semi-quantitative scoring methods to assess the composition of the atherosclerotic plaque and determine its instability following its surgical removal. However, these methods are based on visual estimation without quantitative measurements, which represents a biased approach due to their subjectivity and limited accuracy due to the inter-individual variability. Moreover, these methods are time consuming and require researchers to have previous knowledge in vascular pathology or to rely on vascular pathologists, rendering this technique widely inaccessible. Over the past decade, dramatic improvements in machine-learning image analysis algorithms have promoted the development of powerful quantitative approaches that reduce pathologic interpretation bias and improve the accuracy of disease diagnosis/prognosis and severity grading. For example, the application of computerized image analysis has improved the ability for reliable prognosis in breast cancer, and has allowed more accurate monitoring of fibrotic changes in different stages of liver disease. However, there are limited quantitative pathology studies in the field of atherosclerosis.

Several imaging modalities have been used to characterize plaque features non-invasively including magnetic resonance imaging (MRI), computed tomography (CT), positron emission tomography (PET) and ultrasound. However, they have several limitations. MRI machines provide the highest resolution, but are expensive and scarce. CT imaging provides a lesser resolution than MRI and utilizes carcinogenic radiation, which may accumulate over time along with other CT procedures the subject may endure. Finally, PET imaging uses a contrast agent that must be ingested by the subject in order to visualize metabolic changes of the tissue, which only provides information for a limited number of plaque features. Ultrasound imaging is an operator-dependent technique that has the lowest resolution of these different medical imaging modalities. However, it is a low-cost and readily available tool used to visualize stenosis of the atherosclerotic plaque.

Ultrasound has traditionally been used to assess atherosclerotic disease in terms of both stenosis and plaque morphology. It is safe, reproducible, relatively inexpensive, widely available, and allows frequent subject monitoring; therefore, it has replaced other imaging modalities in the everyday clinical practice, including the decision for surgical interventions. In recent years, digital image analysis techniques and other computer-aided decision tools have been developed to complement ultrasound and provide an objective characterization of plaque instability. Nevertheless, these image analysis techniques have a major limitation, which is the lack of validation with the gold-standard of plaque stability/instability, i.e., histology). As a result, these techniques have not been widely used in clinical practice. More recently, advanced machine-learning techniques have been successfully used for improved diagnosis of breast and thyroid cancers and liver diseases. The framework behind these machine-learning techniques has also the potential to improve diagnostic accuracy of plaque instability and overcome previous limitations.

Virtual histology intravascular ultrasound (VH-IVUS) is an IVUS-based post-processing plaque characterization technique that uses autoregressive spectral analysis of the primary raw backscattered radiofrequency ultrasound signals to generate a reconstructed color-coded map of plaque composition. The plaque can be color-coded as 4 major components (dense calcium, fibrous tissue, fibro-fatty, and necrotic core), as each plaque component possesses its own spectral signatures. These methods have been described by Nair et al. U.S. Pat. No. 7,074,188 and Vince et al. U.S. Pat. No. 6,200,268. VH-IVUS has several major limitations including dependence on accurate borders and misclassification of certain plaque features. If the border is not accurate, the tissue composition can be either overestimated or underestimated. Thrombus may be misclassified as fibrous or fibrofatty tissues, thus reducing the accuracy of this method in identifying an at-risk plaque. Therefore, more accurate methods are needed to characterize plaque composition from ultrasound imaging.

### SUMMARY

It has been discovered that a plurality of additional plaque features, other than stenosis, contribute to the overall risk of a vascular event. As such, an assessment of the presence of each of these plaque features provides for a more accurate characterisation of a plaque and the risk of rupture. It has been discovered that these plaque features include a combination, if not all, of (1) fibrous tissue or fibrosis or scarring (2) neovascularization or new vessels (3) calcification or calcium (4) inflammation (5) hemorrhage (6) foam cells or macrophage (7) lipid or necrotic or fatty core (8) thrombus, (9) fibrous cap, and 10) plaque area that contribute to the stability/instability of the atherosclerotic plaque and the likelihood of it rupturing and causing a heart attack or stroke.

Due to the number of plaque features to be assessed, an image of vasculature would have to be in sufficiently high resolution to allow for distinguishing each of these features. However, current techniques for providing such a high resolution are usually invasive, costly, timely and/or may impact the health of the subject.

Less-costly and less-invasive procedures, such as ultrasound, that are currently available, do not yield images with the necessary resolution or information to distinguish between a high number of plaque features. The present disclosure describes a solution for deriving a sufficiently-information-rich image from an information-poor image of vasculature (e.g., where the information-poor image is taken by ultrasound).

Image registration is the name for a set of techniques that map images into the same coordinate system. In medical imaging, image registration is commonly used to map an image of the same object taken using different imaging modalities into a singular coordinate system. By registering a higher quality image with a lower quality image, valuable information that is not present in the lower quality information can be transferred. This is especially valuable for Machine Learning image segmentation techniques where higher quality data results in a more accurate model. Recently, image registration of MRI onto CT scans has been used to improve the accuracy of image segmentation in lower quality CT scans.

The present disclosure provides a method and system for detecting histopathological plaque features through the segmentation of different medical imaging modalities for the accurate diagnosis and characterization of stable and unstable atherosclerotic plaques using deep neural network techniques.

In some examples, a histology and ultrasound image are inputted into a trained neural network whereby the image is segmented and then scored for plaque stability. A plurality of image pairs (e.g., histological image and information-poor image) may be provided and used to train the neural network as described herein.

A broad aspect is a method of training a neural network for segmenting an information-poor image to identify a plurality of atherosclerotic plaque features in the information-poor image. The method includes receiving a plurality of image pairs, wherein each image pair comprises an information-poor image of specific vasculature and an information-rich image of the specific vasculature, wherein the information-rich image has been adapted to identify one or more regions showing one or more plaque features; and for each image pair of the plurality of image pairs: performing image registration to map the information-poor image and the information-rich image into a same coordinate system; segmenting the information-poor image as a function of the identified one or more regions of the information-rich image, thereby identifying in the information-poor image the one or more plaque features; and comparing the segmented information-poor image to a ground truth based on the information-rich image to calculate a loss that is back-propagated through the neural network to train the neural network.

In some embodiments, the information-poor image may be anultrasound image.

In some embodiments, the ultrasound image may be an atherosclerotic plaque image.

In some embodiments, the information-rich image may be a histopathology image.

In some embodiments, the histopathological image may be a histopathological atherosclerotic plaque image.

In some embodiments, for an information-poor image of a specific vasculature there may be a number of information-rich images of the specific vasculature, wherein each information-rich image of the number of information-rich images has been segmented to show a single plaque feature of said plurality of plaque features, and wherein there may be a plurality of image pairs including the information-poor image and one of the number of information-rich images, wherein each of the plurality of image pairs may correspond to a plaque feature segmented in the information-rich image of the number of information-rich images, and wherein the number of information-rich images may correspond to a number of the plurality of plaque features, and wherein the segmenting of the information-poor image identifies the single plaque feature in the information-poor image.

In some embodiments, the segmenting of the information-poor image may be performed using binary annotations.

In some embodiments, the performing image registration may include: aligning a position of the information-rich image with the information-poor image; detecting local differences between the information-rich image and the information-poor image; and using non-linear transforms to deform the information-rich image for registration so that the information-rich image more closely matches the information-poor image.

In some embodiments, the aligning may include rotating, scaling and shearing at a global image level.

In some embodiments, the plurality of plaque features may include hemorrhage, neovessels, fibrous cap, calcification, inflammation, thrombus, lipid/lipid core, fibrosis, plaque area and foam cells.

In some embodiments, a resolution of the information-poor image of each image pair of the plurality of image pairs may have been enhanced prior to the registering, the segmenting and the comparing.

Another broad aspect is a method of generating guidance information for assessing a risk of a vascular event in a subject through use of a target information-poor image of tissue vasculature of the subject inputted into a trained deep-neural network. The method includes receiving the information-poor image of the tissue vasculature of the subject; using a neural network that is trained with image pairs comprising mapped information-poor images and information-rich images of a same vasculature, to segment the target information-poor image into a plurality of plaque features captured in the information-poor image by differentiating between plaque and non-plaque pixels of the tissue vasculature, using the trained neural network, to define a plaque area; and segmenting the target information-poor image to identify plaque features in the target information-poor image wherein the segmenting is adapted to identify a plurality of plaque features in the target information-poor image.

In some embodiments, the method may include assigning a weight to each plaque feature identified in the segmented target information-poor image; and defining an overall score for a risk of a vascular event for the subject as a function of the assigned weight for each identified plaque feature.

In some embodiments, the non-plaque pixels may include a lumen and a media or adventitial border.

In some embodiments, a resolution of the information-poor image of the image pair may be enhanced prior to the mapping.

In some embodiments, the received target information-poor image may be an ultrasound image and wherein the information-poor image of the image pair may be an ultrasound image.

In some embodiments, the information-rich image of the image pair may be a histopathology image.

In some embodiments, there may be a plurality of information-poor images of a specific vasculature for the information-rich image of the specific vasculature, wherein there may be a plurality of image pairs of the specific vasculature including, for each image pair of the plurality of image pairs of the specific vasculature, the information-rich image and a respective one of the plurality of information-poor images, resulting in the plurality of information-poor images of the specific vasculature being co-registered with the information-rich image of the specific vasculature.

In some embodiments, at least one information-poor image of the plurality of information-poor images may be a transverse view of the specific vasculature and at least one information-poor image of the plurality of information-poor images may be a longitudinal view of the specific vasculature.

In some embodiments, there may be a plurality of information-rich images of a specific vasculature for the information-poor image of the specific vasculature, wherein there may be a plurality of image pairs of the specific vasculature including, for each image pair of the plurality of image pairs of the specific vasculature, the information-poor image and a respective one of the plurality of information-rich images, resulting in the plurality of information-rich images of the specific vasculature being co-registered with the information-poor image of the specific vasculature.

In some embodiments, at least one information-rich image of the plurality of information-rich images may be a transverse view of the specific vasculature and at least one information-rich image of the plurality of information-rich images may be a longitudinal view of the specific vasculature.

In some embodiments, the plaque features may include a combination of three of more of the following: hemorrhage, neovessels, fibrous cap, calcification, inflammation, thrombus, lipid core, fibrosis, plaque area and foam cells.

In some embodiments, the plaque features may include each of hemorrhage, neovessels, fibrous cap, calcification, inflammation, thrombus, lipid core, fibrosis, plaque area and foam cells.

In some embodiments, the method may include updating the calculated overall risk score based on a subject profile, resulting in a modified overall risk score.

In some embodiments, the method may include transmitting the calculated overall score to a computing device of a medical practitioner that is responsible for the subject.

Another broad aspect is a non-transitory storage medium comprising program code that, when executed by a processor, cause the processor to receive a plurality of image pairs, wherein each image pair comprises an information-poor image of specific vasculature and an information-rich image of the specific vasculature, wherein the information-rich image has been adapted to identify one or more regions showing one or more plaque features; and for each image pair of the plurality of image pairs: perform image registration to map the information-poor image and the information-rich image into a same coordinate system; segment the information-poor image as a function of the identified one or more regions of the information-rich image, thereby identifying in the information-poor image the one or more plaque features; and compare the segmented information-poor image to a ground truth based on the information-rich image to calculate a loss that is back-propagated through the neural network to train the neural network.

Another broad aspect is non-transitory storage medium comprising program code that, when executed by a processor, cause the processor to receive the information-poor image of the tissue vasculature of the subject; use a neural network that is trained with image pairs comprising mapped information-poor images and information-rich images of a same vasculature, to segment the target information-poor image into a plurality of plaque features captured in the information-poor image by: differentiating between plaque and non-plaque pixels of the tissue vasculature, using the trained neural network, to define an area of the plaque; and segmenting the target information-poor image to identify plaque features in the target information-poor image wherein the segmenting is adapted to identify a plurality of plaque features in the target information-poor image.

Another broad aspect is a computing device for training a neural network for segmenting an information-poor image to identify a plurality of atherosclerotic plaque features in the information-poor image. The computing device includes a processor; memory comprising program code that, when executed by the processor, cause the processor to receive a plurality of image pairs, wherein each image pair comprises an information-poor image of specific vasculature and an information-rich image of the specific vasculature, wherein the information-rich image has been adapted to identify one or more regions showing one or more plaque features; for each image pair of the plurality of image pairs: perform image registration to map the information-poor image and the information-rich image into a same coordinate system; segment the information-poor image as a function of the identified one or more regions of the information-rich image, thereby identifying in the information-poor image the one or more plaque features; and compare the segmented information-poor image to a ground truth based on the information-rich image to calculate a loss that is back-propagated through the neural network to train the neural network.

Another broad aspect is a computing device for assessing a risk of a vascular event in a subject through use of an information-poor image of vasculature of the subject inputted into a trained deep-neural network. The computing device includes an input/output interface for receiving the information-poor image; a processor; and memory comprising program code that, when executed by the processor, cause the processor to receive the information-poor image of the tissue vasculature of the subject; use a neural network that is trained with image pairs comprising mapped information-poor images and information-rich images of a same vasculature, to segment the target information-poor image into a plurality of plaque features captured in the information-poor image by differentiating between plaque and non-plaque pixels of the tissue vasculature, using the trained neural network, to define an area of the plaque; and segmenting the target information-poor image to identify plaque features in the target information-poor image wherein the segmenting is adapted to identify a plurality of plaque features in the target information-poor image.

Another broad aspect is a method of training a neural network for segmenting an information-poor image to identify a plurality of atherosclerotic plaque features in the information-poor image. The method includes receiving a plurality of image pairs, wherein each image pair comprises an information-poor image of specific vasculature and an information-rich image of the specific vasculature, wherein the information-rich image has been adapted to identify one or more regions showing one or more plaque features; and for each image pair of the plurality of image pairs: performing image registration to map the information-poor image and the information-rich image into a same coordinate system; segmenting the information-poor image as a function of the identified one or more regions of the information-rich image, thereby identifying in the information-poor image the one or more plaque features; and comparing the segmented information-poor image to a ground truth based on the information-rich image to calculate an error in said identifying in the information-poor image the one or more plaque features with respect to said ground truth that is back-propagated through the neural network to train the neural network.

### BRIEF DESCRIPTION OF THE DRAWINGS

The invention will be better understood by way of the following detailed description of embodiments of the invention with reference to the appended drawings, in which:

FIG. 1A is a block diagram of an exemplary system architecture for assessing the risk of a vascular event based on plaque analysis.

FIG. 1B is a block diagram of an exemplary computing system for characterizing the composition of exemplary plaques captured in information-poor images.

FIG. 2 illustrates an exemplary workflow schematic of creating an overall risk score using deep neural networks.

FIG. 3 illustrates an exemplary workflow schematic of training a neural network using registered histopathology images.

FIG. 4 Illustrates an example of image registration.

FIG. 5 illustrates an exemplary deep convolutional neural network.

FIG. 6 illustrates exemplary histopathological plaque features to be extracted.

FIG. 7 is a diagram of a U-NET machine learning model utilizing a VGG16 architecture for segmentation of plaque features in a histological image using a neural network.

FIG. 8 illustrates a series of histological images on the leftmost of the figure for training a neural network, followed by expected segmentation of the plaques identified in the images into plaque features by an expert (ground truth), and followed by the segmentation of the plaques of the histological images into plaque features performed by the neural network (prediction).

FIG. 9 illustrates an exemplary diagram for, at the top of the diagram, detecting one or more plaques in a low-resolution image (an ultrasound image) using a trained neural network, and at the bottom of the diagram, segmenting the plaques into plaque features using the trained neural network.

FIG. 10A is an ultrasound image of a longitudinal view of vasculature with a plaque that has been segmented into plaque features using a trained neural network.

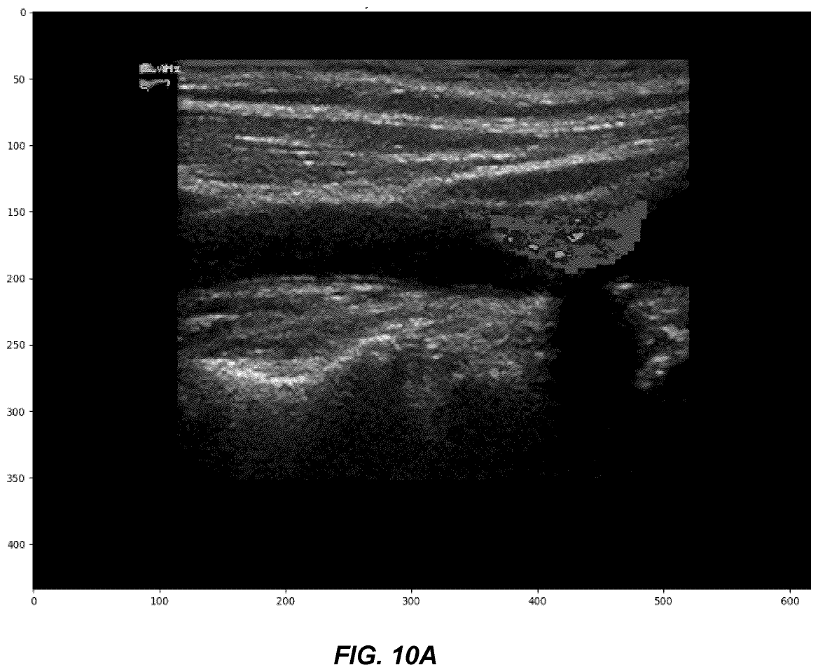

FIG. 10B is an ultrasound image of a transverse view of vasculature with a plaque that has been segmented into plaque features using a trained neural network.

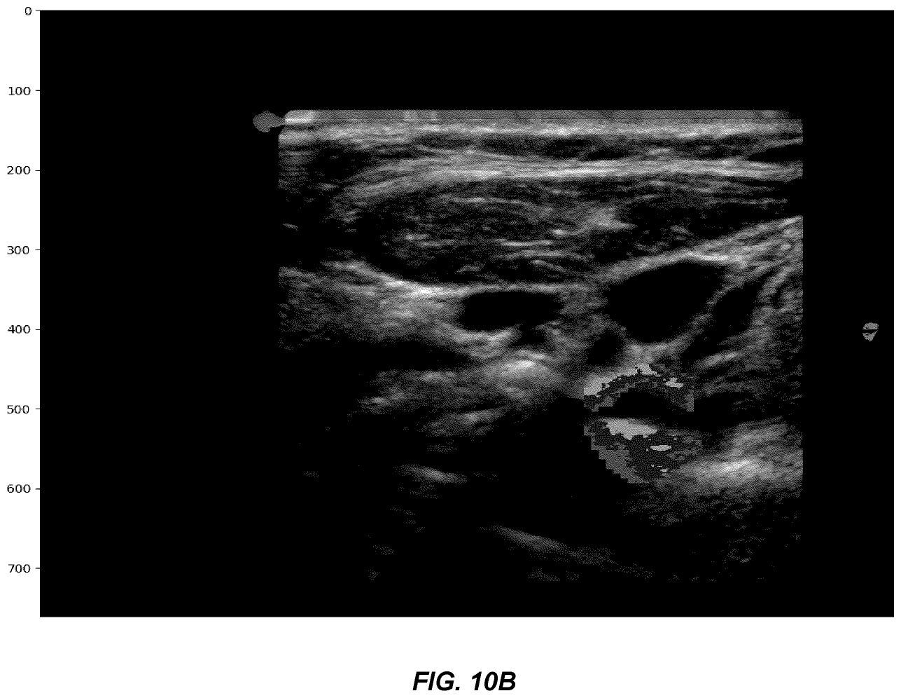

### DETAILED DESCRIPTION

The present disclosure relates to a system and method for providing guidance information for assessing a risk of a vascular event (heart attack or stroke) from an information-poor image of vasculature of a subject, and then using a trained neural network to enhance the resolution or information of the image such that plaque features can be identified and analyzed.

A risk score of a vascular event caused by the plaque based on the analyzed enhanced image can then be generated in order to provide an indicator to the medical practitioner as to the severity of the plaque and the risk of a vascular event. In addition to the information obtained from the analyzed medical image, other subject factors (termed “subject profile”), including demographics, clinical characteristics, other imaging data, blood biomarkers, and “omics” data (e.g., genomics, epigenomics, transcriptomics, proteomics, metabolomics, lipidomics, etc.), among others may be used to refine the overall risk score assessment.

### Definitions

In the present disclosure, by “guidance information”, it is meant information that can be used by a medical practitioner to assist with an assessment or diagnosis of a subject. The guidance information may, in some embodiments, suggest a diagnosis for a subject.

In the present disclosure, by “information-rich image”, it is meant an image that is used for the purpose of enhancing information found in an information-poor image, such as by increasing the resolution of the information-poor image, such as the plaque features are more discernable. For instance, an information-rich image may be a histological image. In some cases, the information-rich images used may include transverse views, longitudinal views or a combination of transverse and longitudinal views of vasculature.

In the present disclosure, by “information-poor image”, it is meant an image that undergoes an information enhancement using an information-rich image, whereby enhancing information increases the discernability of one or more of the plaque features appearing in the image. For instance, an information-poor image may be an ultrasound image. In some cases, the information-poor images used may include transverse views, longitudinal views or a combination of transverse and longitudinal views of vasculature.

In the present disclosure, by “medical practitioner”, it is meant a doctor, a nurse, a healthcare professional, a medical equipment technician, a medical researcher, etc.

In the present disclosure, by “subject”, it is meant a mammal, such as a human, a pig, a dog, etc. The term “subject” should not bring on any limitations as to the sex or age, or race/ethnicity. A subject may be undergoing a regular follow-up or check-up with its medical practitioner.

In the present disclosure, by “subject profile”, it is meant characteristics of the subject such as demographics, clinical characteristics, other imaging data, blood biomarkers, and “omics” data (e.g., genomics, epigenomics, transcriptomics, proteomics, metabolomics, lipidomics, etc.). The subject profile may be used to refine the overall risk score assessment

### Exemplary Architecture of a System for Generating Guidance Information for Assessing the Risk of a Vascular Event Based on Plaque Analysis

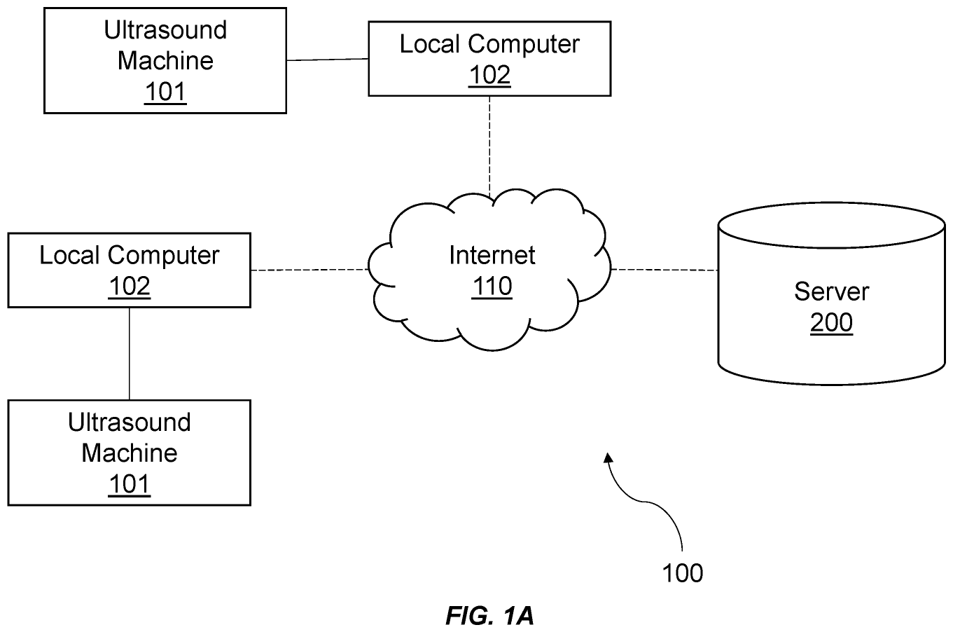

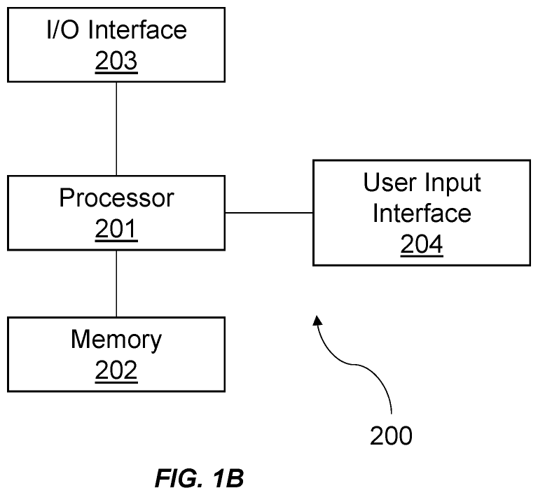

Reference is made to FIG. 1A, illustrating an exemplary architecture of a system 100 for generating guidance information for assessing the risk of a vascular event in a subject based on plaque analysis.

The system 100 includes one or more information-poor image generators, such as an ultrasound machine 101, that are connected to one or more local computers 102 (e.g., used by a medical practitioner). For purpose of illustration, when discussing an information-poor image generator, the example of an ultrasound machine 101 will be used. However, it will be understood that other machines for obtaining information-poor images of a subject's tissue (including plaques) may be used without departing from the present teachings including IVUS, low contrast resolution CT, MRI, PET imaging, etc.

The system 100 also includes one or more servers 200, connected to the local computers 102 over the Internet 110.

The ultrasound machine 101 is used to generate one or more ultrasound images of a subject, namely of the vasculature of a subject, including the presence of any atherosclerotic plaques, where an intima-media thickness may be determined from the one or more ultrasound images. The ultrasound images provide information on one or more plaques that can be used to derive plaque morphology. However, the resolution of the ultrasound images is not sufficient to identify a plurality of plaque features with sufficiently high accuracy, as described herein. As such, the ultrasound images undergo a resolution enhancement through registration to a histological image in order to allow for a more precise analysis of the plaque features found in the plaque of the ultrasound images.

The generated ultrasound images are transferred via the local computer 102, over the Internet 110, to the server 200.

The server 200 includes program code for a trained neural network, receiving the ultrasound image and performing a transfer of information to the information-poor image, thereby enhancing the resolution of the low-resolution ultrasound image. Following the enhancement of the resolution of the ultrasound image, the enhanced ultrasound images are then analyzed at the server 200 to define each plaque feature, assess the severity of each plaque feature, and assign a score to each plaque feature representative of the severity of the plaque feature. An overall score may also be generated as to the risk of a vascular event that can be suffered by the subject, where the overall score may be generated from the scores produced for each plaque feature. Additional subject factors, indicated as subject profile, can also be included in the overall risk score, such as demographics, clinical characteristics, other imaging data, blood biomarkers, and “omics” data (e.g., genomics, epigenomics, transcriptomics, proteomics, metabolomics, lipidomics, etc.), among others.

The ultrasound image with the enhanced resolution, the plaque feature scores, and/or the overall score of a vascular event may be sent to the local computer 102, via the Internet 110, enabling, e.g., a medical practitioner to have access to the information for, e.g., providing information to assist in clinical decision-making (e.g., to decide if a surgical intervention is warranted to remove the plaque, or if medications are to be administered).

Reference is now made to FIG. 1B, illustrating an exemplary server 200 for receiving one or more ultrasound images, enhancing the resolution of the one or more ultrasound images using a trained neural network stored in memory, segmenting the image based on plaque feature, assigning scores to the plaque features and/or assigning an overall score as to the risk of a vascular event.

The server 200 includes a processor 201, memory 202 (where the memory 202 may be non-transitory) and an input/output interface 203.

The server 200 may include one or more user input interfaces 204.

The processor 201 may be a general-purpose programmable processor. In this example, the processor 201 is shown as being unitary, but the processor may also be multicore, or distributed (e.g., a multi-processor).

The computer readable memory 202 stores program instructions and data used by the processor 201. The memory 202 may be non-transitory. The computer readable memory 202, though shown as unitary for simplicity in the present example, may comprise multiple memory modules and/or cashing. In particular, it may comprise several layers of memory such as a hard drive, external drive (e.g., SD card storage) or the like and a faster and smaller RAM module. The RAM module may store data and/or program code currently being, recently being or soon to be processed by the processor 201 as well as cache data and/or program code from a hard drive. A hard drive may store program code and be accessed to retrieve such code for execution by the processor 201 and may be accessed by the processor 201 to store low-resolution ultrasound images, image pairs of information-poor images and information-rich images, ultrasound images of enhanced resolution, as explained herein. The memory 202 may have a recycling architecture for storing, for instance, low-resolution ultrasound images, image pairs of information-poor images and information-rich images, ultrasound images of enhanced resolution, etc., where older data files are deleted when the memory 202 is full or near being full, or after the older data files have been stored in memory 202 for a certain time.

The input/output (I/O) interface 203 is in communication with the processor 201. The I/O interface 203 is a network interface and may be a wireless interface for establishing a remote connection with, for example, a remote server, an external database, etc. For instance, the I/O interface 203 may be an Ethernet port, a WAN port, a TCP port, etc.

The processor 201, the memory 202 and the I/O interfaces 203 may be linked via BUS connections.

The user input interface 204 is for allowing a user to provide input to the server 200 in order to interact with the server 200. The user input interface 204 may be a mouse 105, keyboard 106 and/or controller 107 and may be used to receive user input from the user.

It will be understood that other user input interfaces may be used in accordance with the present teachings, such as a touchscreen, a joystick, a microphone, one or more proximity sensor detecting movement of the user, etc.

### Exemplary Method of Generating an Overall Risk Score of a Vascular Event

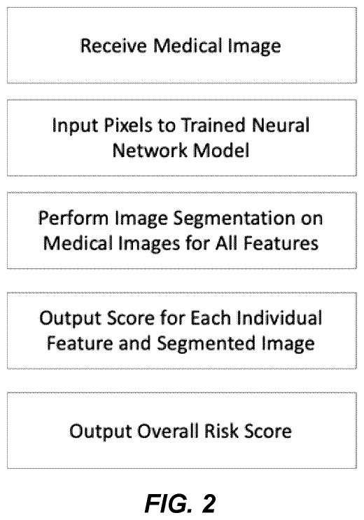

Reference is now made to FIG. 2 illustrating an exemplary method of generating an overall risk score for a plaque using a non-invasive medical image that is generated at a low resolution but enhanced by the present system. The medical image is received by the system, and afterwards its pixels are input into a trained neural network. The neural network performs image segmentation and selects the parts of the image that belong to each feature. Given the segmentation, a score for each feature is calculated and then a composite score is generated from each of the feature scores. Additional subject factors, indicated as subject profile, can also be included in the overall risk score, such as demographics, clinical characteristics, other imaging data, blood biomarkers, and “omics” data (e.g., genomics, epigenomics, transcriptomics, proteomics, metabolomics, lipidomics, etc.), among others.

### An Exemplary Method of Training a Neural Network for Enhancing the Information of a Low-Resolution Medical Image

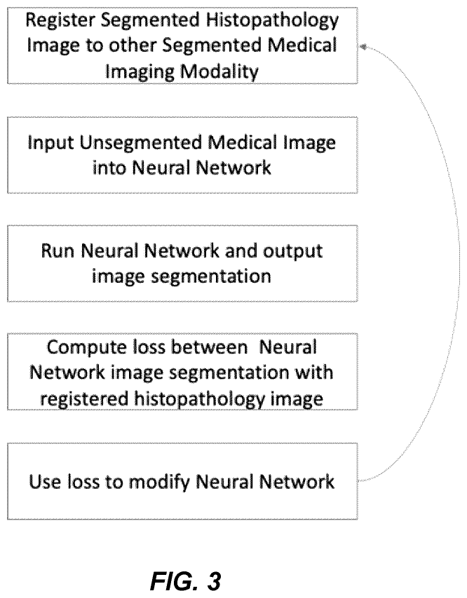

Reference is now made to FIG. 3, illustrating an exemplary method of training a neural network using paired images where one image is a higher quality (information-rich) medical image and the other is a lower quality (information-poor) non-invasive medical image that will be used for inference and diagnostic. In some instances, for a same information-poor image of vasculature, there may be a plurality of information-rich images of the same vasculature (forming respectively a plurality of image pairs with the same information-poor image of the vasculature) that can be co-registered with the information-poor image. In some instances, for a same information-rich image of vasculature, there may be a plurality of information-poor images of the same vasculature (forming respectively a plurality of image pairs with the same information-rich image of the vasculature) that can be co-registered with the information-rich image.

The neural network is trained by registering the higher resolution, or information-rich, histopathology image with the lower, information-poor, resolution image, where valuable information from the higher quality image is transferred to the coordinate system of the lower quality image. Once this information has been transferred, a higher quality neural network model can be trained.

For training of the neural network, the histopathology images may be segmented by an expert before training of the neural network begins. Experts examine the histopathology image and highlight which parts of the image correspond to which plaque feature. The highlighted parts of the image can be converted into a binary image, where I represents where the plaque feature is and O represents where it is not, or the plaque feature can be described as a categorical, ordinal, or continuous variable. The segmentation of the image may be repeated on separate images for each plaque feature. In some examples, techniques may be used to employ an information-rich image that is unsegmented for the purpose of registration with the low-resolution/information-poor image.

When training the system, the system is provided with pairs of different images of the same tissue: an information-poor image (e.g., ultrasound image) with the annotated equivalent histopathology image, indicating the presence of a plaque feature (information-rich)—a plurality of annotated equivalent histopathology images may be provided, one for each plaque feature. In some examples, a histopathological image may be annotated with a plurality of plaque features. In some embodiments, an annotated equivalent histopathology image may indicate the presence of a plurality of plaque features, depending on the annotation system.

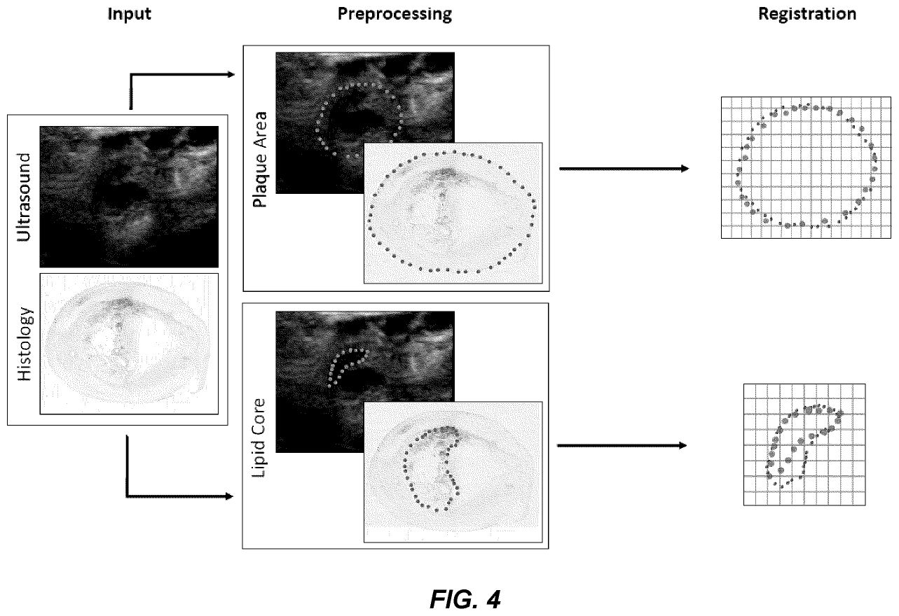

In the example of the method presented in FIG. 3, the segmented histopathology image is registered onto the information-poor image (it will be understood that the information-poor image may have been segmented by an expert prior to the registration of the segmented histopathology image; in other examples, the information-poor image may be unsegmented prior to the registration). An exemplary embodiment of image registration is provided in FIG. 4. The registration accounts for differences in the images due to imaging techniques and time variations. The information-poor image is then segmented to determine a plaque feature, with reference to the segmented histopathology image, where values are attributed to objects in the information-poor image to indicate the presence, or degree of presence (e.g., intensity) or absence of the plaque feature.

In some instances, the system may determine from image pairs of information-rich images and information-poor images plaque features that are present on both information-rich images and information-poor images without prior annotation (for purposes of segmentation to identify the plaque features) from an expert.

FIG. 4 illustrates an example of non-rigid image registration. The histopathology image that is going to be registered, is aligned using translation, rotation, scaling and shearing at a global image level. In some instances, once the images are aligned, small local differences between both images are detected and non-linear transforms are used to deform the image that will be registered so that it more closely matches the lower resolution image.

The example of FIG. 4 shows two image pairs that are marked-up to identify a plaque area and a lipid core, respectively, in each of the ultrasound image and the histopathological image. Even though FIG. 4, for purpose of illustration, refers to lipid core and plaque features as identified features, it will be appreciated that other plaque features may be identified and/or registered in accordance with the present teachings. The histopathological image has been marked-up (e.g., by an expert) to define a plaque area, as shown in the top image, and a lipid core as shown in the bottom image. Following a mapping of the information-poor image with the information-rich image in each image pair, the low-resolution image is segmented to further identify respectively, for each image pair, the plaque area and the lipid core. The information-rich and information-poor images, once the features identified, are then registered with respect to one another (e.g., with a mapping of the images).

At the beginning of training, the neural network is initialized randomly and the training process iteratively changes the network so that it makes gradually improved predictions. A neural network takes an input, runs the input through it and produces an output. This output is then compared to a ‘ground truth’, a loss is calculated by comparing the output of the neural network with the ground truth, and this loss is back-propagated through the neural network to update the neural network.

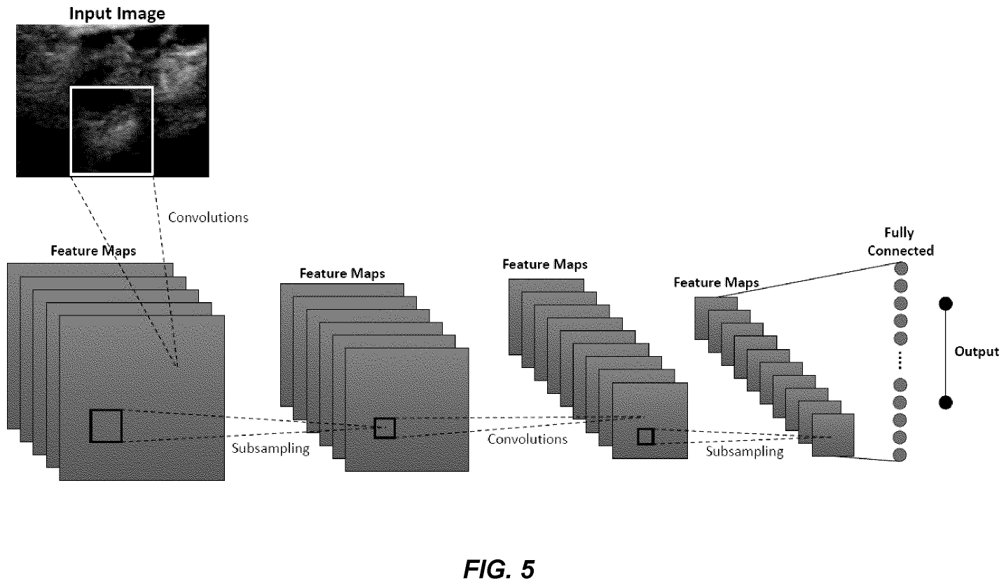

The lower quality image is used as input to a deep convolutional neural network (DCNN). The architecture of the DCNN may be a convolutional, batch normalization, subsampling layers (e.g., pooling layers) with a feedforward layer in the end as shown in FIG. 5. A convolutional block may be composed of a convolutional, and subsampling layers together. In some embodiments, the convolutional block may also include batch normalization. Many convolutional blocks may be stacked to increase the predictive power of the network. Between each convolutional block there may be a non-linear activation function. Traditionally these are Rectified Linear Units but others can be used. Two to three convolutional blocks can be stacked to make up a residual block, each residual block has a connection that adds the input to the residual block to the output of the residual block, which helps in training the DCNN.

The DCNN outputs 12 binary images that are the same size as the input image. In one example, each image represents one plaque feature, and the values are 0 if the plaque feature was not present in that pixel and 1 if it was. A loss is then computed by comparing the output of the DCNN with the registered histopathology image. The information from the registered histopathology image provides richer information that improves the loss, which guides the neural network to update it in a better manner.

This training enables the system to detect certain plaque features in information-poor images as a result of the image being paired with information-rich images that have been marked-up by an expert during training, the trained system being sufficiently sensitive to derive plaque information present in an information-poor image, such as that obtained through ultrasound, to identify up to 12 different kinds of plaque features, as a result of the training. The ultrasound images to be diagnosed can then be fed to the system for the purpose of identifying a high plurality of plaque features.

FIG. 5 Illustrates a deep convolutional neural network. The network takes the pixel values of the image and applies convolutions, matrix multiplication and nonlinear functions in a feed-forward manner. Combinations of these operations constitute a layer, and the network is many layers deep. The aforementioned operations are exemplary, and do not represent all of the mathematical operations that can make up a layer.

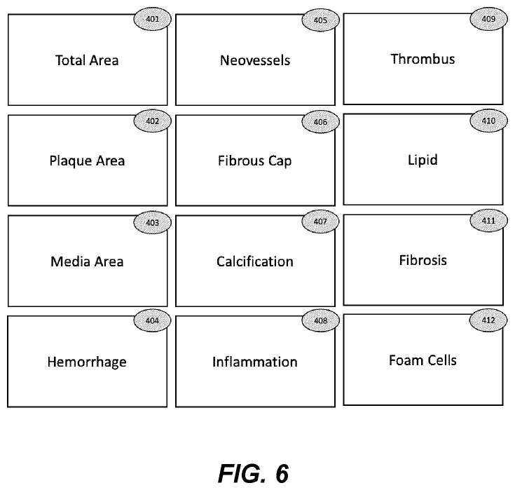

FIG. 6 illustrates the exemplary 12 features that can be identified by the trained neural network from the histopathology image. In some embodiments, in order to improve accuracy of the score predicting the risk of a vascular event, all twelve features can be identified. The total tissue area 401 (representing pixels of the plaque and the non-plaque area, i.e. the media) will be differentiated into, the plaque area 402 of the histopathology image and the medial layer 403. The plaque area 402 comprises plaque features 404 to 412 (hemorrhage, neovessels, fibrous cap, calcification, inflammation, thrombus, lipid core, fibrosis, and foam cells). The area of the media 403 is calculated and may be defined as the area between the adventitia and intimal layers, made up of smooth muscle cells between two layers of elastic lamina. The extent of hemorrhage 404, defined as the rupturing of blood through a blood vessel, is characterized by analyzing the red-purple pigment in the pixel identified as red blood cells within the plaque, as well as the density of those pixels. The size and number of neovessels 405, defined as the formation of new blood vessels within the plaque, are quantified. The fibrous cap 406, defined as the extent of fibrotic connective tissue between the arterial lumen and the underlying matrix. The area of the fibrous cap and the thickness between the arterial lumen and underlying matrix are quantified. The total area of vascular calcification 407 and number of calcified nodules, defined as the deposition of calcium in the plaque, is identified. The extent of intraplaque inflammation 408 is analyzed by quantifying the area, number, intensity, and density of the stained inflammatory cells (represented by stained pixels) after immunohistochemistry with antibodies (including but not limited to CD68,MACI, MAC2, CD3, CD20, smooth-muscle α-actin,), identifying inflammatory cells such as macrophages, lymphocytes, smooth muscle cells, fibroblasts, among others. The area of the thrombus 409, defined as the presence of a blood clot near the lumen of the plaque, is quantified. A thrombus is generally formed by the rupture of a plaque and the leakage of its contents into the arterial lumen. The lipid area 410, defined as the presence of a necrotic lipid core made up of foam cells, cholesterol crystals, and dead cells, is quantified and analyzed. The extent of fibrosis 411, not including fibrosis found in the fibrous cap, is identified. Fibrosis is defined as the proliferation, migration and thickening of smooth muscle cells into the intimal and medial layer. The number, size and extent of foam cell generation 412 in the atherosclerotic plaque are analyzed by quantifying the area, intensity, and density of the stained foam cells (represented by stained pixels) after immunohistochemistry with antibodies identifying foam cells.

Each identified plaque feature is assigned a score or weight based on the severity of the plaque feature with respect to its possible contribution to a vascular event.

An overall score of a risk of a vascular event arising from the plaque (e.g. heart attacks, strokes) for the given subject, based on the segmented and analyzed ultrasound image of the subject's vasculature, and the plaque, is generated from the scores and/or weights assigned to each plaque feature.

Additional characteristics, that may be part of a “subject profile” listing history and/or characteristics (such as comorbidities of the subject) and/or biomarkers, may be added to the models for an accurate determination of the heart attack or stroke risk, including but not limited to artery stenosis, subject demographics (age, sex, gender, race/ethnicity, etc.), anthropometric measurements (e.g., weight, height, waist circumference, hip circumference, etc.) clinical data (e.g., blood pressure, lipid profile, blood glucose, etc.) family history, reproductive history, comorbidities (e.g., obesity, cardiovascular disease, previous atherosclerotic lesions or vascular event, diabetes mellitus, hypertension, dyslipidemia, etc.), lifestyle habits (e.g., smoking, alcohol consumption, physical activity, etc.), medications, psychosocial factors, other imaging data, blood biomarkers, and other “omics” data (e.g., genomics, epigenomics, transcriptomics, proteomics, metabolomics, lipidomics, etc.), among others. These additional characteristics of the subject may be used to refine the overall risk score, where for example, presence of risk factors would change the score to indicate a higher risk of a vascular event, and where absence of risk factors or presence of protective factors would change the score to indicate a lower risk of vascular event, resulting in a modified overall risk score, taking into account these additional characteristics of the “subject's profile”.

In some instances, the plaque analysis described herein from the information-poor images, using the trained neural network, may be used to determine a percentage of stenosis or lumen narrowing of vasculature of a subject. The result may be output as a value (e.g., a fraction or percentage or area reduction) indicative of the proportion of the vasculature that is blocked by a plaque, or that is unobstructed by a plaque.

In some instances, the plaque analysis described herein from the information-poor images, using the trained neural network, may be used to calculate an intima-media thickness of vasculature, an early stage of atherosclerosis, indicative of plaque development and risk of cardiovascular events.

In some instances, the plaque analysis described herein from the information-poor images, using the trained neural network and the plurality of information-poor images (transverse and longitudinal), may be used to generate a three-dimensional model of the plaque appearing in the image-poor image(s).

In some instances, the plaque analysis described herein from the information-rich images, using the trained neural network and the plurality of information-rich images (transverse and longitudinal), may be used to generate a three-dimensional model of the plaque appearing in the image-rich image(s).

### Exemplary Studies

The following exemplary studies are provided to enable the skilled person to better understand the present disclosure. As they are but illustrative and representative examples, they should not limit the scope of the present disclosure, only added for illustrative and representative purposes. It will be understood that other exemplary studies may be used to further illustrate and represent the present disclosure without departing from the present teachings.

### Exemplary Study 1

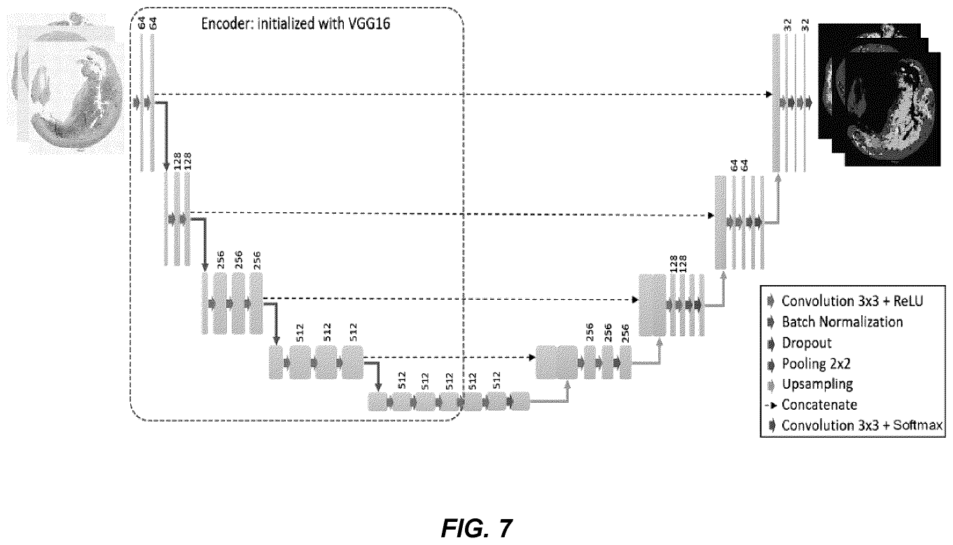

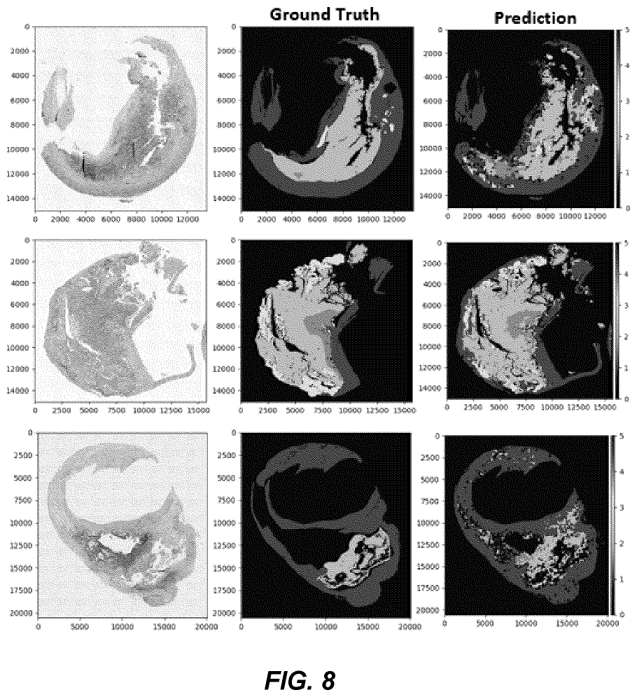

Semantic segmentation of major plaque features. of an atherosclerotic plaque from histopathology images was performed, e.g., fibrosis, lipid core, calcification, media, hemorrhage, thrombus, fibrous cap, neovascularization using a Convolutional Neural Network (CNN) and U-Net model. For the encoder part of the U-Net model several backbone architectures pre-trained on ImageNet dataset were examined, and the VGG16 was selected (FIG. 7). Models were trained using training samples (histological images) and evaluated using the validation and unseen test datasets. In the example presented in FIG. 8, ‘Ground truth’ represents the annotations of the different components of the plaque provided by an expert, and ‘Prediction’ represents the prediction of the plaque components by the U-Net model.

### Exemplary Study 2

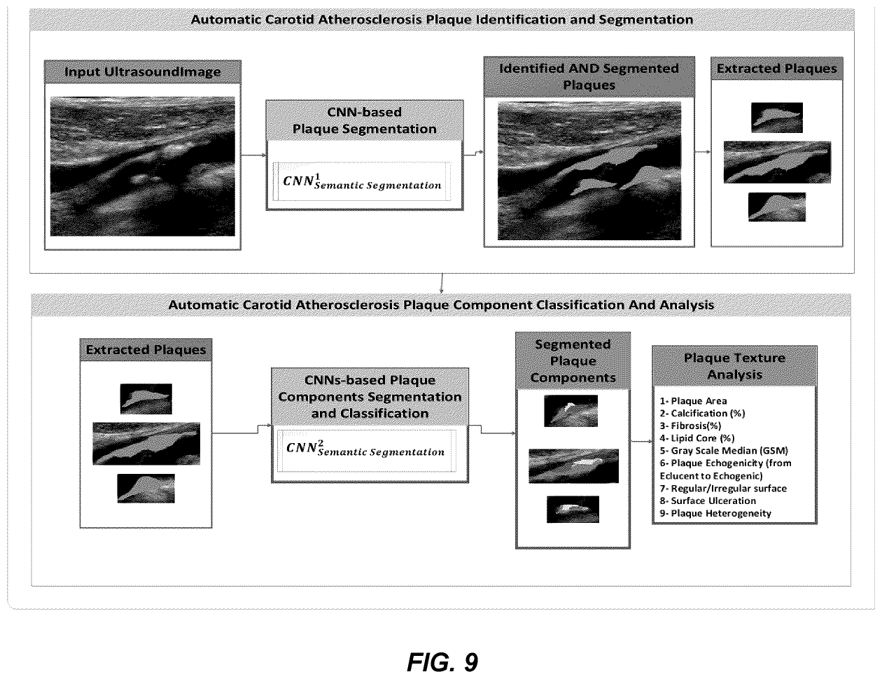

A fully automated ultrasound computer-assisted diagnosis system for atherosclerosis plaque characterization incudes two portions: 1) plaque detection and segmentation, 2) plaque feature segmentation and classification, focusing on analyses of certain plaque features, including gray scale median (median of the intensity values of the pixels inside the plaque) to assess plaque echogenicity, plaque thickness, plaque area, degree of stenosis or luminal narrowing (%), fibrosis, lipid core, calcification, texture heterogeneity, surface ulceration, regular/irregular plaque surface, as illustrated for instance at FIG. 9, 10A and 10B.

Automatic anonymization and automatic masking of ultrasound images are followed by automatic standard normalization of these images. For plaque detection and segmentation, a CNN-based semantic segmentation model with U-net architecture was constructed. For the encoder part of the U-Net model several backbone architectures, pre-trained on ImageNet dataset, were examined and the ResNet34 was selected. Different rotations, translations, scaling, and different intensity variations (brightness) were used to augment the variations of the training dataset. Automatic image processing techniques were used to automatically calculate: 1) plaque thickness, using Principal Component Analysis technique, 2) plaque area, 3) plaque volume based on both corresponding longitudinal and transverse images, 4) lumen diameter to calculate stenosis (lumen narrowing) and severity of stenosis (moderate and severe), as well as 5) pixel-based analyses and segmentation of the plaque features, as mentioned above. Both longitudinal and transverse B-mode images were analyzed and colour and Dopler images were used as an aid. Models were trained using the training histological samples and evaluated using the validation and unseen test datasets. An automated CNN-and machine learning-based model was constructed for stenosis classification and tested on longitudinal images.

Although the invention has been described with reference to preferred embodiments, it is to be understood that modifications may be resorted to as will be apparent to those skilled in the art. Such modifications and variations are to be considered within the purview and scope of the present invention.

Representative, non-limiting examples of the present invention were described above in detail with reference to the attached drawings. This detailed description is merely intended to teach a person of skill in the art further details for practicing preferred aspects of the present teachings and is not intended to limit the scope of the invention. Furthermore, each of the additional features and teachings disclosed above and below may be utilized separately or in conjunction with other features and teachings.

Moreover, combinations of features and steps disclosed in the above detailed description, as well as in the experimental examples, may not be necessary to practice the invention in the broadest sense, and are instead taught merely to particularly describe representative examples of the invention. Furthermore, various features of the above-described representative examples, as well as the various independent and dependent claims below, may be combined in ways that are not specifically and explicitly enumerated in order to provide additional useful embodiments of the present teachings.

What is claimed is:

1. A method of training a neural network for segmenting an information-poor image to identify a plurality of atherosclerotic plaque features in the information-poor image comprising:
receiving a plurality of image pairs, wherein each image pair comprises an information-poor image of specific vasculature and an information-rich image of the specific vasculature, wherein the information-rich image has been adapted to identify one or more regions showing one or more plaque features; and
for each image pair of the plurality of image pairs:
performing image registration to map the information-poor image and the information-rich image into a same coordinate system;
segmenting the information-poor image as a function of the identified one or more regions of the information-rich image, thereby identifying in the information-poor image the one or more plaque features; and
comparing the segmented information-poor image to a ground truth based on the information-rich image to calculate a loss that is back-propagated through the neural network to train the neural network.

2. The method as defined in claim 1, wherein the information-poor image is an ultrasound image.

3. The method as defined in claim 2, wherein the ultrasound image is an atherosclerotic plaque image.

4. The method as defined in any one of claims 1 to 3, wherein the information-rich image is a histopathology image.

5. The method as defined in claim 4, wherein the histopathological image is a histopathological atherosclerotic plaque image.

6. The method as defined in any one of claims 1 to 5, wherein for an information-poor image of a specific vasculature there is a number of information-rich images of the specific vasculature, wherein each information-rich image of the number of information-rich images has been segmented to show a single plaque feature of said plurality of plaque features, and wherein there is a plurality of image pairs including the information-poor image and one of the number of information-rich images, wherein each of the plurality of image pairs corresponds to a plaque feature segmented in the information-rich image of the number of information-rich images, and wherein the number of information-rich images corresponds to a number of the plurality of plaque features, and wherein the segmenting of the information-poor image identifies the single plaque feature in the information-poor image.

7. The method as defined in claim 6, wherein the segmenting of the information-poor image is performed using binary annotations or categorical, ordinal or continuous values.

8. The method as defined in any one of claims 1 to 7, wherein there is a plurality of information-poor images of a specific vasculature for the information-rich image of the specific vasculature, wherein there is a plurality of image pairs of the specific vasculature including, for each image pair of the plurality of image pairs of the specific vasculature, the information-rich image and a respective one of the plurality of information-poor images, resulting in the plurality of information-poor images of the specific vasculature being co-registered with the information-rich image of the specific vasculature.

9. The method as defined in claim 8, wherein at least one information-poor image of the plurality of information-poor images is a transversal view of the specific vasculature and at least one information-poor image of the plurality of information-poor images is a longitudinal view of the specific vasculature.

10. The method as defined in any one of claims 1 to 7, wherein there is a plurality of information-rich images of a specific vasculature for the information-poor image of the specific vasculature, wherein there is a plurality of image pairs of the specific vasculature including, for each image pair of the plurality of image pairs of the specific vasculature, the information-poor image and a respective one of the plurality of information-rich images, resulting in the plurality of information-poor images of the specific vasculature being co-registered with the information-rich image of the specific vasculature.

11. The method as defined in claim 10, wherein at least one information-rich image of the plurality of information-rich images is a transversal view of the specific vasculature and at least one information-rich image of the plurality of information-rich images is a longitudinal view of the specific vasculature.

12. The method as defined in any one of claims 1 to 11, wherein the performing image registration comprises:
aligning a position of the information-rich image with the information-poor image;
detecting local differences between the information-rich image and the information-poor image; and
using non-linear transforms to deform the information-rich image for registration so that the information-rich image more closely matches the information-poor image.

13. The method as defined in claim 12, wherein the aligning comprising rotating, scaling and shearing at a global image level.

14. The method as defined in any one of claims 1 to 13, wherein the plurality of plaque features includes hemorrhage, neovessels, fibrous cap, calcification, inflammation, thrombus, lipid/lipid core, fibrosis, plaque area and foam cells.

15. The method as defined in any one of claims 1 to 14, wherein a resolution of the low-resolution image of each image pair of the plurality of image pairs has been enhanced prior to the registering, the segmenting and the comparing.

16. A method of generating guidance information for assessing a risk of a vascular event in a subject through use of a target information-poor image of tissue vasculature of the subject inputted into a trained deep-neural network, comprising:
receiving the information-poor image of the tissue vasculature of the subject;
using a neural network that is trained with image pairs comprising mapped information-poor images and information-rich images of a same vasculature, to segment the target information-poor image into a plurality of plaque features captured in the information-poor image by:
differentiating between plaque and non-plaque pixels of the tissue vasculature, using the trained neural network, to define a plaque area; and
segmenting the target information-poor image to identify plaque features in the target information-poor image wherein the segmenting is adapted to identify a plurality of plaque features in the target information-poor image.

17. The method of claim 16, further comprising:
assigning a weight to each plaque feature identified in the segmented target information-poor image; and
defining an overall score for a risk of a vascular event for the subject as a function of the assigned weight for each identified plaque feature.

18. The method as defined in claim 17, further comprising updating the calculated overall score based on a “subject profile”, resulting in a modified overall score.

19. The method as defined in claim 16 or claim 18, wherein the non-plaque pixels include a lumen and a media or adventitial border.

20. The method as defined in any one of claims 16 to 19, wherein a resolution of the information-poor image of the image pair is enhanced prior to the mapping.

21. The method as defined in any one of claims 16 to 20, wherein the received target information-poor image is an ultrasound image and wherein the information-poor image of the image pair is an ultrasound image.

22. The method as defined in any one of claims 16 to 21, wherein the information-rich image of the image pair is a histopathology image.

23. The method as defined in any one of claims 16 to 22, wherein the plaque features include a combination of three of more of the following: hemorrhage, neovessels, fibrous cap, calcification, inflammation, thrombus, lipid core, fibrosis, plaque area and foam cells.

24. The method as defined in any one of claims 16 to 23, wherein the plaque features include each of hemorrhage, neovessels, fibrous cap, calcification, inflammation, thrombus, lipid core, fibrosis, plaque area and foam cells.

25. The method as defined in any one of claims 16 to 24, further comprising transmitting the calculated overall score to a computing device of a medical practitioner that is responsible for the subject.

26. A non-transitory storage medium comprising program code that, when executed by a processor, cause the processor to:
receive a plurality of image pairs, wherein each image pair comprises an information-poor image of specific vasculature and an information-rich image of the specific vasculature, wherein the information-rich image has been adapted to identify one or more regions showing one or more plaque features; and
for each image pair of the plurality of image pairs:
perform image registration to map the information-poor image and the information-rich image into a same coordinate system;
segment the information-poor image as a function of the identified one or more regions of the information-rich image, thereby identifying in the information-poor image the one or more plaque features; and
compare the segmented information-poor image to a ground truth based on the information-rich image to calculate a loss that is back-propagated through the neural network to train the neural network.

27. A non-transitory storage medium comprising program code that, when executed by a processor, cause the processor to:
receive the information-poor image of the tissue vasculature of the subject;
use a neural network that is trained with image pairs comprising mapped information-poor images and information-rich images of a same vasculature, to segment the target information-poor image into a plurality of plaque features captured in the information-poor image by:
differentiating between plaque and non-plaque pixels of the tissue vasculature, using the trained neural network, to define an area of the plaque; and
segmenting the target information-poor image to identify plaque features in the target information-poor image wherein the segmenting is adapted to identify a plurality of plaque features in the target information-poor image.

28. A computing device for training a neural network for segmenting an information-poor image to identify a plurality of atherosclerotic plaque features in the information-poor image, comprising:
a processor;
memory comprising program code that, when executed by the processor, cause the processor to:
receive a plurality of image pairs, wherein each image pair comprises an information-poor image of specific vasculature and an information-rich image of the specific vasculature, wherein the information-rich image has been adapted to identify one or more regions showing one or more plaque features;
for each image pair of the plurality of image pairs:
perform image registration to map the information-poor image and the information-rich image into a same coordinate system;
segment the information-poor image as a function of the identified one or more regions of the information-rich image, thereby identifying in the information-poor image the one or more plaque features; and
compare the segmented information-poor image to a ground truth based on the information-rich image to calculate a loss that is back-propagated through the neural network to train the neural network.

29. A computing device for assessing a risk of a vascular event in a subject through use of an information-poor image of vasculature of the subject inputted into a trained deep-neural network, comprising:
an input/output interface for receiving the information-poor image;
a processor; and
memory comprising program code that, when executed by the processor, cause the processor to:
receive the information-poor image of the tissue vasculature of the subject;
use a neural network that is trained with image pairs comprising mapped information-poor images and information-rich images of a same vasculature, to segment the target information-poor image into a plurality of plaque features captured in the information-poor image by:
differentiating between plaque and non-plaque pixels of the tissue vasculature, using the trained neural network, to define an area of the plaque; and
segmenting the target information-poor image to identify plaque features in the target information-poor image wherein the segmenting is adapted to identify a plurality of plaque features in the target information-poor image.

30. A system for generating guidance information for assessing a risk of a vascular event in a subject through use of a target information-poor image of tissue vasculature of the subject inputted into a trained deep-neural network, the system comprising:
a client device for receiving the information-poor image of the tissue vasculature of the subject;
a server operatively connected to the client device for receiving the information-poor image from the client device and comprising a processor and memory comprising program code that, when executed by the processor, cause the processor to:
use a neural network that is trained with image pairs comprising mapped information-poor images and information-rich images of a same vasculature, to segment the target information-poor image into a plurality of plaque features captured in the information-poor image by:
differentiating between plaque and non-plaque pixels of the tissue vasculature, using the trained neural network, to define a plaque area;
segment the target information-poor image to identify plaque features in the target information-poor image wherein the segmenting is adapted to identify a plurality of plaque features in the target information-poor image; and
communicate to said client device data concerning said plaque features in the target information-poor image.

31. The system as defined in claim 30, wherein the information-poor image is an ultrasound image and the information-rich image is obtained from histological analysis.
